(19) United States
(12) Patent Application Publication
(10) Pub. No.: US 2014/0041673 A1
(43) Pub. Date: Feb. 13, 2014

(54) DEVICES FOR CONTAINING FLUID AND DEBRIS ON ENDOTRACHEAL TUBES, LARYNGEAL MASKS, ENDOSCOPES, BRONCHOSCOPES AND CATHETERS AND METHOD OF USE THEREOF

(71) Applicant: Christopher C. Walters, Carlsbad, CA (US)

(72) Inventor: Christopher C. Walters, Carlsbad, CA (US)

(21) Appl. No.: 13/966,040

(22) Filed: Aug. 13, 2013

Related U.S. Application Data
(60) Provisional application No. 61/742,463, filed on Aug. 13, 2012.
(60) Provisional application No. 61/718,347, filed on Oct. 25, 2012.

(51) Int. Cl. A61B 17/12

(52) U.S. Cl. CPC A61B 17/12104
USPC 128/887

(57) ABSTRACT

Devices, methods and kits for reducing the likelihood of exposure to bodily fluid from a patient during the process of intubation and/or extubation of a tubular surgical device. Some of the bodily fluid guards can be used in conjunction with previously existing devices, while others are designed to be integrated with devices during manufacture of the devices. The bodily fluid guards has opposite superior and inferior ends with a wall that is configured to capture fluid and debris from the tubular surgical device, thereby preventing the bodily fluid and debris from contacting the patient, caregivers and surrounding equipment. In some embodiments, the bodily fluid guard includes a flexible, extendible sleeve wall to contain or substantially contain the device during extubation, thereby capturing any fluid and debris present on the device, wherein the wall can be releasably fixed to the device and to the patient to facilitate use.

### CROSS-REFERENCE TO RELATED APPLICATION

This application claims the benefit of U.S. Provisional Application Ser. No. 61/742,463, filed Aug. 13, 2012, and the benefit of U.S. Provisional Application Ser. No. 61/718,347, filed Oct. 25, 2012, both of which are incorporated herein by reference in their entirety.

### BACKGROUND OF THE INVENTION

1. Technical Field

This invention relates generally to devices that improve the function and safety of endotracheal tubes (ETTs), laryngeal masks (LMs), bronchoscopes, and catheters.

2. Related Art

Patients' mouths, throats and airways can contain mucous, phlegm, saliva, blood, pus and plaque, any of which can act as a vehicle for virus, fungi and bacteria. When an ETT, LM, catheter, or bronchoscope is introduced into or removed from a patient, these bodily fluids and/or tissue fragments can be flung into the immediate surroundings, thereby potentially exposing anyone near the patient, such as physicians, nurses, caregivers, patients in nearby beds and potentially family members to the patient's bodily fluids and any pathogens they might contain. The bodily fluids and debris can also contaminate surrounding equipment in the operating theatre, such as anesthesia machines, work surfaces, patient monitors, and the like. Thus, there is clearly a need in the art for improved ETT, LM, bronchoscope and catheter devices that can reduce or eliminate the possibility of contamination with a patient's bodily fluids during extubation.

### SUMMARY OF THE INVENTION

In accordance with one aspect of the invention, a bodily fluids guard for containing fluid and debris on a breathing tube of a laryngeal mask, endotracheal tube device, bronchoscope, or a catheter extubated from a patient is provided. The bodily fluids guard includes a flexible tubular wall extending between an open superior end and an open inferior end and further, at least one inferior end fastener extending from the inferior end. The at least one fastener has a surface adapted for releasable fixation to a patient.

In accordance with another aspect of the invention, the bodily fluids guard can further include at least one superior end fastener extending from the superior end, with the at least one superior end fastener having a surface adapted for fixation to the breathing tube.

In accordance with another aspect of the invention, the at least one inferior end fastener and said at least one superior end fastener can be provided as self-adhesive surfaces.

In accordance with another aspect of the invention, the at least one inferior end fastener can be provided to seal the open inferior end.

In accordance with another aspect of the invention, the inferior end fastener can be provided as a zip lock member.

In accordance with another aspect of the invention, the inferior end fastener can be provided as a draw string.

In accordance with another aspect of the invention, the bodily fluids guard can be provided having a first annular ring of semi-rigid material adjacent the superior end, wherein the at least one superior end fastener extends from the first annular ring.

In accordance with another aspect of the invention, the bodily fluids guard can include a second annular ring of semi-rigid material adjacent the inferior end, wherein the at least one inferior end fastener extends from the second annular ring.

In accordance with another aspect of the invention, the first annular ring can be provided having a reduced diameter relative to the second annular ring.

In accordance with another aspect of the invention, the first annular ring can be provided having a cone region extending radially outwardly therefrom toward the second annular ring.

In accordance with another aspect of the invention, the first annular ring and second annular ring can be releasably coupled to one another.

In accordance with another aspect of the invention, a bodily fluids guard can include a superior end; an inferior end; an outer layer; an inner layer having a hollow core; wherein the inner and outer layers are connected to one another and the inner layer includes a compressible and absorbent material, and the outer layer includes a flexible material, wherein the hollow core is configured to receive a breathing tube of the LM device, ETT device, or bronchoscope, or a catheter.

In accordance with another aspect of the invention, bristles can extend radially inwardly into the hollow core to facilitate cleaning the breathing tube.

In accordance with another aspect of the invention, at least one grasping feature can be provided to extend radially outward from the outer layer to facilitate grasping and holding the bodily fluids guard in its intended location during extubation.

In accordance with another aspect of the invention, a method of containing fluid and debris on a breathing tube of a LM, ETT, bronchoscope, or catheter device being extubated from a patient is provided. The method includes providing a flexible tubular wall extending between an open superior end and an open inferior end in a lengthwise collapsed state; disposing the a tube through a core of the collapsed tubular wall; intubating the tube into the patient with the collapsed tubular wall placed in abutment with the patient; releasably fastening the inferior end of the collapsed tubular wall to the patient; extubating the tube from the patient and simultaneously lengthening the tubular wall to surround the tube.

In accordance with another aspect of the invention, the tube can be provided as a breathing tube.

In accordance with another aspect of the invention, the method can further include fastening the superior end to the tube prior to extubating.

In accordance with another aspect of the invention, the method can further include fastening the inferior end to the patient with a self-adhesive and fastening the superior end to the tube with a self-adhesive.

In accordance with another aspect of the invention, the method can further yet include sealing the open inferior end closed after extubating the tube with a fastener adjacent the open inferior end.

### BRIEF DESCRIPTION OF THE DRAWINGS

These and other aspects, features and advantages of the present invention will become more readily appreciated when considered in connection with the following detailed description of presently preferred embodiments and best mode, appended claims and accompanying drawings, in which:

FIG. 1A is a perspective view of a bodily fluids guard constructed in accordance with one aspect of the invention;

FIG. 1B is a perspective view of a bodily fluids guard constructed in accordance with another aspect of the invention;

FIGS. 2A-2C illustrate a bodily fluid guard constructed in accordance with one aspect of the invention against a patient's skin with a breathing tube operably received therein;

FIG. 3A is a perspective view of a bodily fluids guard constructed in accordance with another aspect of the invention;

FIG. 3B is a plan view of the bodily fluids guard of FIG. 3A;

FIG. 3C is a perspective view of the bodily fluids guard of FIG. 3A shown being disposed on a patient;

FIG. 3D is a side view of the bodily fluids guard of FIG. 3C upon disposing a breathing tube therethrough;

FIG. 4A is a perspective view of a bodily fluids guard constructed in accordance with another aspect of the invention;

FIG. 4B is a bottom view of the bodily fluids guard of FIG. 4A;

FIGS. 4C-4E show progressive views of the bodily fluid guard of FIG. 4A in use;

FIG. 5A is a perspective view of a bodily fluids guard constructed in accordance with another aspect of the invention;

FIG. 5B is a side view of the bodily fluids guard of FIG. 5A shown disposed on a patient with a breathing tube disposed therein;

FIG. 6A is a side view of a bodily fluids guard constructed in accordance with another aspect of the invention shown in use against a patient with a breathing tube disposed therethrough;

FIG. 6B is a front view of the bodily fluids guard of FIG. 6A;

FIG. 6C is a front view of the bodily fluids guard of FIGS. 6A and 6B shown in use with the breathing tube retracted and sealed therein for disposal;

FIG. 7A is a side view of a bodily fluids guard constructed in accordance with another aspect of the invention shown in use against a patient with a breathing tube disposed therethrough;

FIG. 7B is a side view of the bodily fluids guard of FIG. 7A shown in use with a breathing tube retracted and sealed therein for disposal;

FIG. 8A is a perspective view of a bodily fluids guard constructed in accordance with another aspect of the invention shown in a collapsed state prior to receiving a breathing tube therethrough;

FIG. 8B shows the bodily fluids guard of FIG. 8A with an extubated breathing tube retracted and sealed therein for disposal after intubation and use of the breathing tube;

FIG. 9A is a perspective view of a bodily fluids guard constructed in accordance with another aspect of the invention shown in a collapsed state prior to use;

FIG. 9B shows a plan view of the bodily fluids guard of FIG. 9A;

FIG. 9C shows the bodily fluids guard of FIG. 9A with an extubated breathing tube fully retracted and sealed therein for disposal;

FIG. 10A is a side view of a bodily fluids guard constructed in accordance with another aspect of the invention shown with a breathing tube received therethrough;

FIG. 10B is a side view of the bodily fluids guard of FIG. 10A shown in an extended state with the breathing tube fully retracted and sealed therein for disposal;

FIG. 11A is a side view of a packaged bodily fluids guard constructed in accordance with another aspect of the invention; (CFI embodiment)

FIG. 11B is top view of the bodily fluids guard of FIG. 11A with the outer packaging removed therefrom and with a secondary tubular containment packaging extending thereabout;

FIG. 11C is a side view of the bodily fluids guard of FIG. 11B;

FIG. 11D is a side view of the bodily fluids guard of FIG. 11B with a breathing tube disposed therethrough;

FIG. 11E is a side view of the bodily fluids guard of FIG. 11B with the secondary tubular packaging removed therefrom and with the guard secured about the mouth of a patient and to the breathing tube;

FIG. 11F is a side view of the bodily fluids guard of FIG. 11E shown in an extended state with the breathing tube fully retracted therein;

FIG. 11G is a side view of the bodily fluids guard of FIG. 11F shown sealed therein for disposal after use;

FIG. 12 is an extended side view of the bodily fluids guard constructed in accordance with another aspect of the invention shown with a breathing tube retracted and sealed therein for disposal after use;

FIG. 13A is a perspective view of a bodily fluids guard constructed in accordance with another aspect of the invention shown in a partially expanded state;

FIG. 13B is a perspective view of the bodily fluids guard of FIG. 13A shown in use against a patient with a breathing tube disposed therethrough;

FIG. 13C is a perspective view of the bodily fluids guard of FIG. 13A with the breathing tube shown retracted and sealed therein for disposal after use;

FIG. 14A is a perspective view of a bodily fluids guard constructed in accordance with another aspect of the invention shown in a collapsed state;

FIG. 14B is a side view of the bodily fluids guard of FIG. 14A shown in use against a patient with a breathing tube disposed therethrough;

FIG. 14C is a side view of the bodily fluids guard of FIG. 14B with the breathing tube shown retracted and sealed therein for disposal after use;

FIG. 15A is a perspective view of a bodily fluids guard shown integrated as an assembly with a tubular device in accordance with another aspect of the invention;

FIG. 15B is a view similar to FIG. 15A with a protective wrapper removed from the bodily fluids guard;

FIG. 15C is side view of a patient with the tubular device intubated with a patient and the bodily fluids guard secured about the mouth of the patient;

FIG. 15D is a side view of the bodily fluids guard of FIG. 15C shown in an extended state with the tubular device fully retracted therein; and

FIG. 15E is a side view of the bodily fluids guard of FIG. 15D shown sealed therein for disposal after use.

### DETAILED DESCRIPTION OF PRESENTLY PREFERRED EMBODIMENTS

All references cited herein are incorporated by reference in their entirety as though fully set forth. Unless defined otherwise, technical and scientific terms used herein have the same meaning as commonly understood by one of ordinary skill in the art to which this invention belongs. Lippincott Williams & Wilkins, Clinical Anesthesia, 6th Edition (2009) provides one skilled in the art with a general guide to many of the terms used in the present application.

In view of this disclosure, one skilled in the art will recognize many methods and materials similar or equivalent to those described herein, which could be used in the practice of the present invention. Indeed, the present invention is in no way limited to the methods and materials described herein.

“Mammal” as used herein refers to a member of the class Mammalia, including, without limitation, humans as well as nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, newborn subjects and infant subjects, whether male or female, are intended to be included within the scope of this term.

In some embodiments, the numbers expressing units of measurement, used to describe and claim certain embodiments of the application are to be understood as being modified by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

As used herein, the acronym “BFG” means bodily fluids guard.

As used herein, the acronym “LMA” means laryngeal mask airway, which is the most common type of laryngeal mask (LM) currently used, and is referred to generically herein as breathing tube.

As used herein, the acronym “ETT” means endotracheal tube, and is referred to generically herein as breathing tube.

There are two general categories of breathing tube devices predominantly used by medical professionals to channel gas (eg. air, anesthetic, etc.) into a patient's lungs. The first type of device, a LM, has an airway tube, also referred to as breathing tube, that connects to an elliptical mask with a cuff. When the cuff is inflated, the mask conforms to the patient's anatomy, with the bowl of the mask facing the space between the vocal cords. When correctly inserted, the tip of the laryngeal mask sits in the throat against the muscular valve that is located at the upper portion of the esophagus. The second type of device most commonly used is a tracheal tube, also referred to herein as breathing tube. A tracheal tube is a catheter that is inserted into the trachea in order to establish and maintain a patient's airway and to ensure the adequate exchange of various gases, such as oxygen and carbon dioxide. Many different types of tracheal tubes are available, and suited for different applications. An ETT is a specific type of tracheal tube that is either inserted through the mouth (orotracheal) or nose (nasotracheal). Another type of tube used by physicians to view an airway or lungs is referred to as bronchoscope, also referred to herein as generally as breathing tube.

As indicated above, perhaps one of the greatest problems with commercially available LMs and ETTs is that they all create a risk of exposing caregivers and others, as well as equipment and surfaces to a patient's bodily fluids during intubation and/or extubation. Recognizing the importance of this issue, the inventor designed a number of types of BFGs that are considerably improved in reducing the risk of exposing caregivers and others, including the patient, and equipment and surfaces to the patient's bodily fluids during intubation and/or extubation. These BFGs can be separated into two general categories, those that can be used in conjunction with previously existing models of ETTs, LMs, bronchoscopes and catheters, and those that are integrated into entirely new ETTs, LMs, bronchoscopes and catheters. Both categories of devices are included in the ensuing description in addition within the drawings submitted herewith.

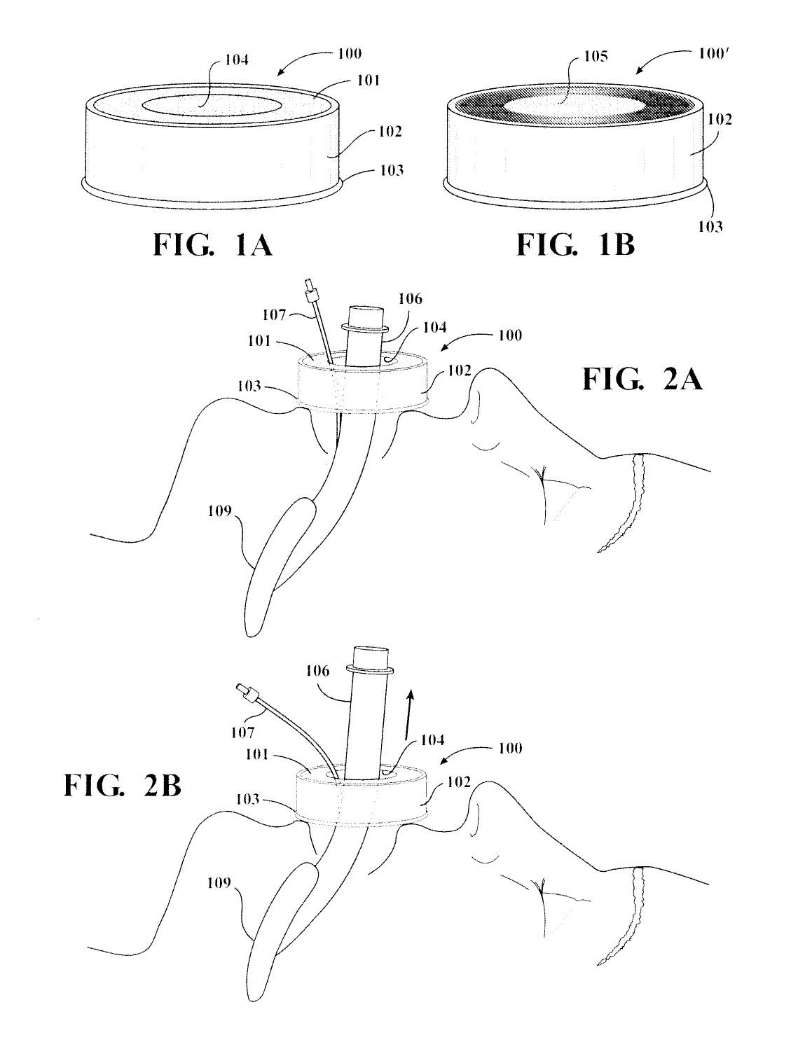

Referring in more detail to the drawings, FIG. 1A depicts, in accordance with an embodiment of the invention, a bodily fluids guard (BFG) 100 with an approximately cylindrical hollow core 104 formed in a sponge material 101, which is in turn circumscribed by an outer layer of flexible, compressible plastic 102 with a stabilizing ridge 103 that extends radially outwardly from the plastic outer layer 102 on an inferior end of the BFG 100. One of skill in the art would readily appreciate that the sponge-like material 101 can be any compressible material with or without absorbent properties. One of skill in the art would also readily appreciate that the device could be any number of shapes that are compatible with its intended purpose. Merely by way of example, the device could be round or elliptical in shape. FIG. 1B depicts, in accordance with an embodiment of the invention, a slightly modified version of the BFG 100 shown in 1A, designated by 100′, with the added feature of bristles 105 lining the interior of the bodily fluids guard 100′ and pointing to the approximate center of the hollow core.

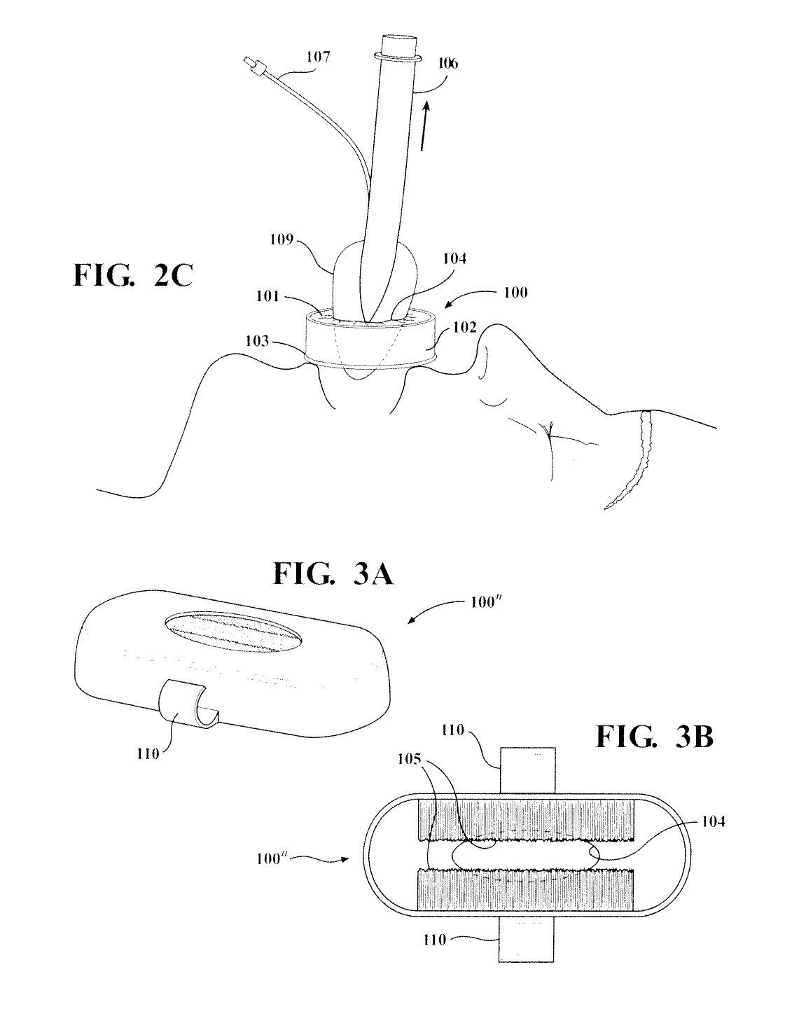

FIG. 2A depicts, in accordance with an embodiment of the invention, the BFG 100 fitted over a breathing tube 106 portion of a breathing tube device 108, shown as a LM device, by way of example and without limitation. The head (cuff) 109 of the LM device can be seen, along with a tube 107 for inflating the head 109. FIG. 2B depicts the LM device 108 of FIG. 2A extracted approximately half way out of a patient. FIG. 2C depicts bodily fluids and tissue debris being removed from the head 109 of the LM device 108 via frictional engagement of the head 109 with the material 101 bounding the core 104 as it is retracted through the BFG 100. In some embodiments, the BFG 100 is made of a material that can be compressed during extubation to apply greater pressure against the breathing tube 106 and/or the head 109 of the LM device 108, in order to more readily and effectively remove bodily fluids and other contaminants therefrom.

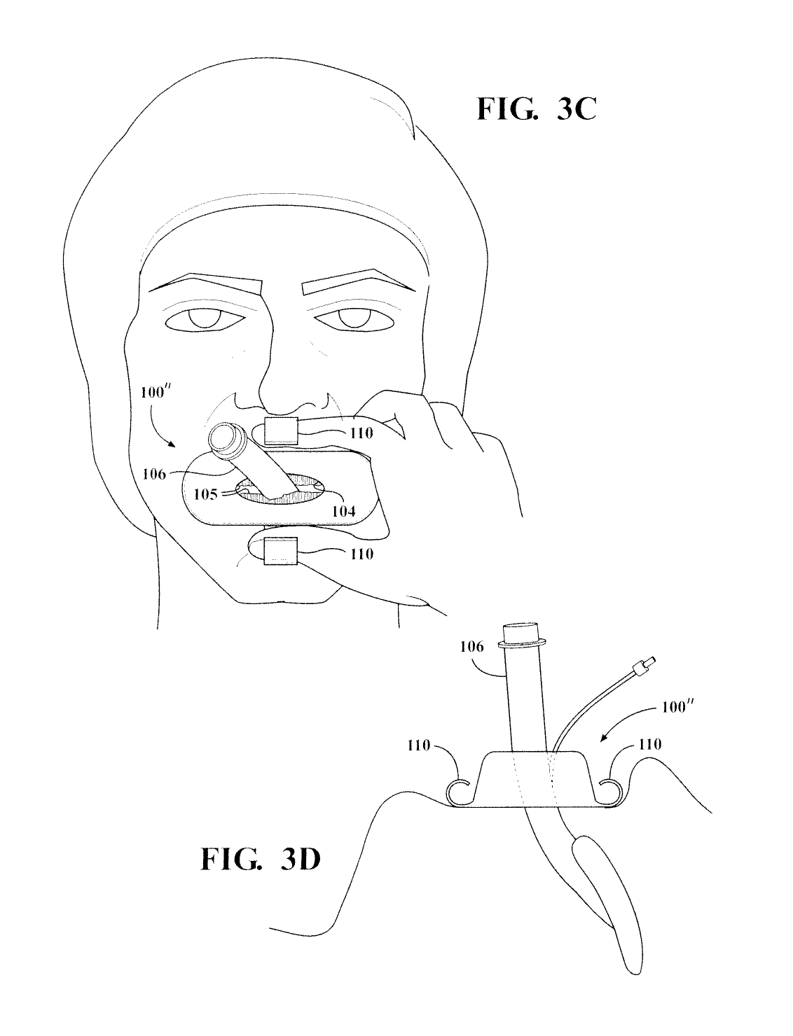

FIG. 3A depicts, in accordance with an embodiment of the invention, a perspective view of a version of a BFG 100″ with grasping features extending radially outwardly from the outer layer 102, shown as being in the form of loops 110, by way of example, attached to the sides of the BFG 100″ and configured for insertion of a medical caregiver's fingers, in order to provide greater leverage and therefore greater stability during extubation (removal of the breathing tube from the patient). FIG. 3B depicts a bottom view of the bottom surface of the BFG 100″. The opening 104 that allows an LM or ETT device to pass through the BFG 100″ can be seen in this view, with bristles 105 shown extending radially inwardly for removal of bodily fluids and debris during extubation. FIG. 3C depicts the device 100″ positioned over a breathing tube 106 and against a patient's mouth, with a caregiver's fingers threaded through the finger loops 110, and FIG. 3D depicts a side view of the device 100″ against the patient.

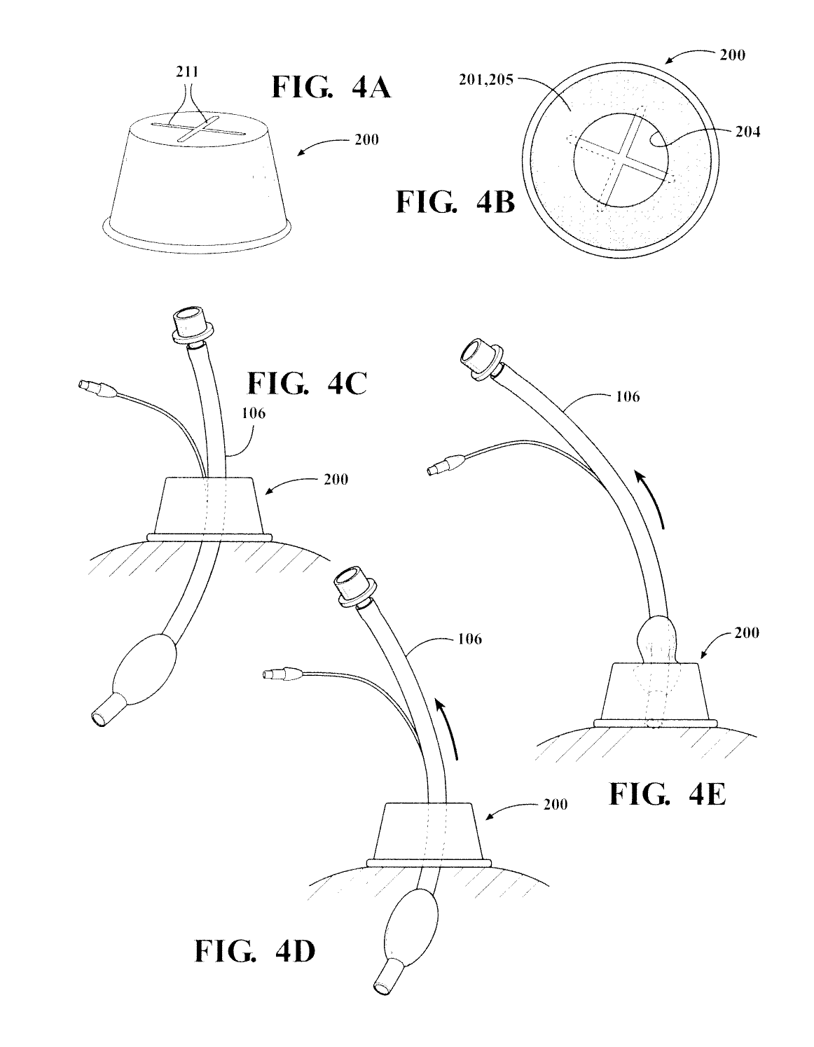

FIGS. 4A and 4B depict perspective and bottom views, respectively, of a bodily fluids guard (BFG) 200 constructed in accordance with another embodiment of the invention. FIGS. 4C-4E depict a breathing tube 106, shown as an ETT, by way of example and without limitation, being progressively retracted through the BFG 200 and be cleaned via sponge material 201 and/or bristles 205 lining the core 204. The BFG 200 is generally cone-shaped, being frustroconical, with slits 211 on the superior end of the BFG 200.

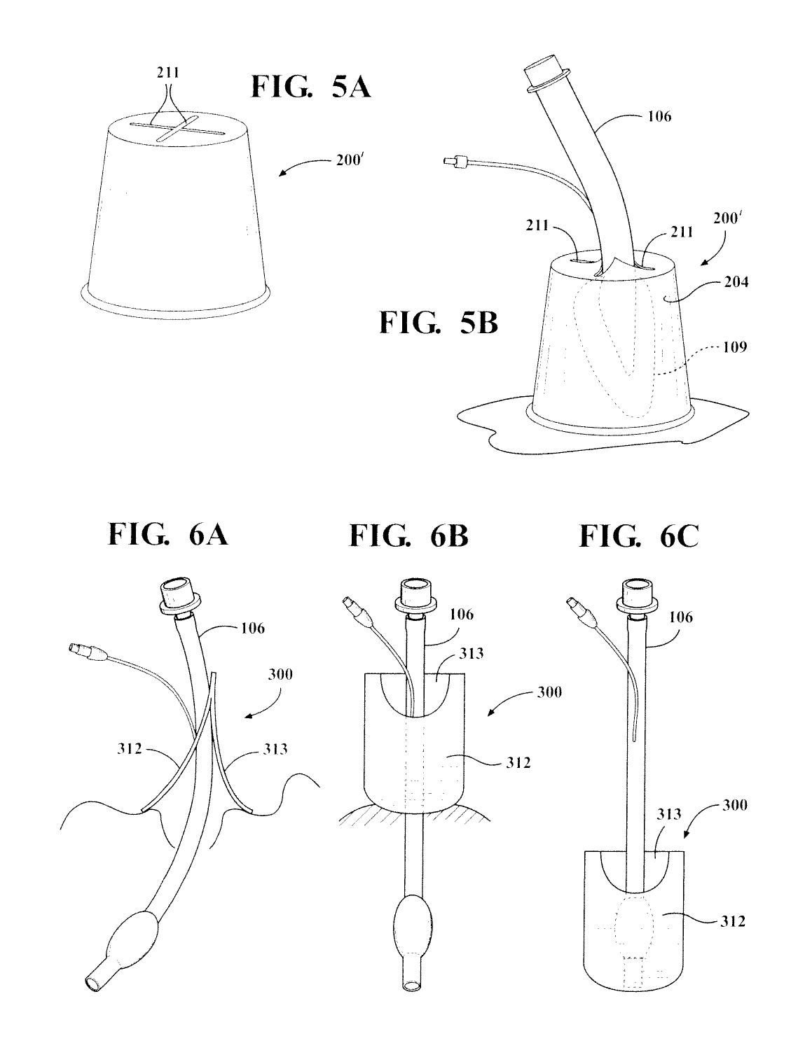

FIG. 5A depicts, in accordance with an embodiment of the invention, a “cup” type BFG device 200′ with slits 211, similar as shown in FIG. 4. However, in FIG. 5B it can be seen that the cup type BFG device 200′, as the breathing tube 106, such as that of an LM device, by way of example and without limitation, is pulled through he slits 211 on the reduced diameter tapered superior end of the device 200′, the head 109 of the LM device 106 can be fully retracted within the cone-shaped core 204 the BFG device 200′, thereby being fully contained therein.

FIG. 6A depicts, in accordance with an embodiment of the invention, a side view of a “duck bill” type BFG device 300. The wall of the BFG device 300 is preferably formed from a thin flexible material that is shown spread out and somewhat conforming relation to the patient's face. FIG. 6B depicts, in accordance with an embodiment of the invention, a front view of the BFG device 300 in which a flexible front flap 312 can be seen. As the breathing tube 106 is removed from the patient (extubated), the device 300 remains in contact with the patient's face. FIG. 6C depicts a front view of the inferior tip of a breathing tube apparatus, such as an LM, by way of example and without limitation, enclosed within the duck bill-shaped BFG device 300. As shown, the inferior ends of the front and rear flaps 312, 313 of the BFG device 300 are configured to stick or bond together beyond the inferior end of an LM or ETT device, and can be further facilitated to stick together via incorporation of a fastener, such as an adhesive, by way of example.

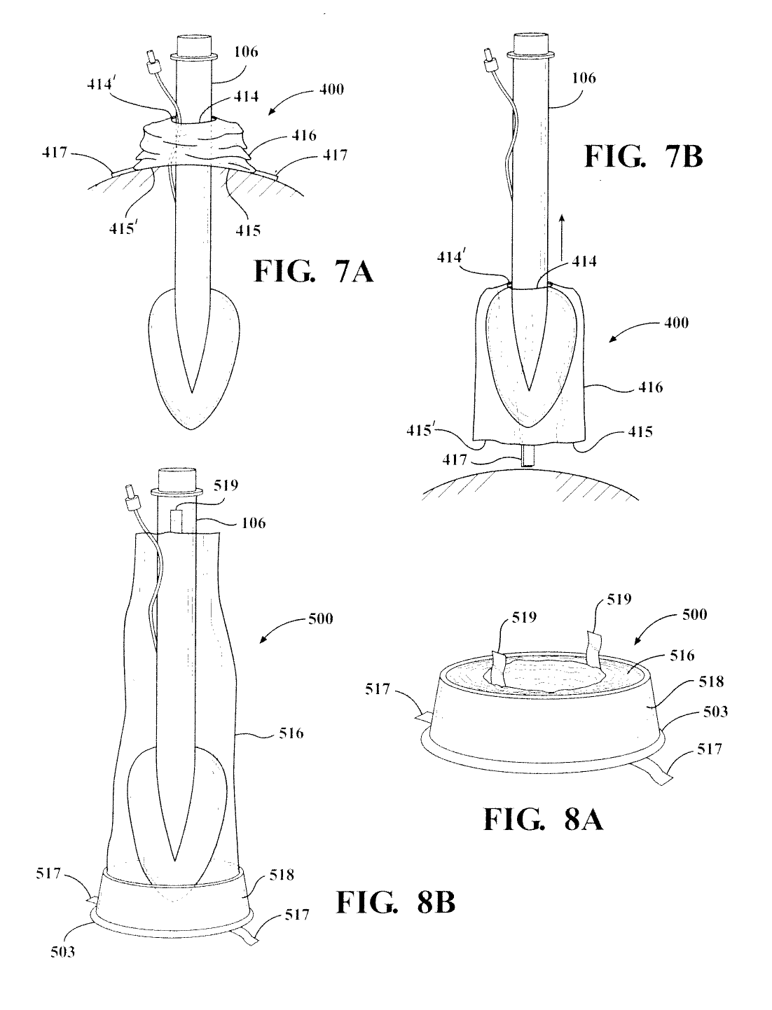

FIG. 7A depicts, in accordance with another embodiment of the invention, a “sleeve” or “bag” type BFG 400 with a breathing tube 106, such as of a ETT or an LM device, extending through an opening 414, 415 in each superior and inferior ends 414′, 415′ of a flexible tubular sleeve wall 416. At least one, and shown as a plurality of inferior end fasteners having a surface adapted for releasable fixation to a patient, such as self-adhesive members having release tape thereon, also referred to as tags or wings 417, are provided at one end, also referred to as an inferior end 415′, to releasably fix the inferior end 415′ of the sleeve wall 416 to a patient, such as the patient's face, while the head 109 of the breathing tube 106 is positioned inside of the patient's mouth. Of course, prior to releasably fixing the tags 417 to the patient, the release paper is removed therefrom to expose a self-adhesive face of the tags 417. FIG. 7B depicts the inferior end of the breathing tube device being completely withdrawn from the patient's mouth and inside the sleeve wall 416 of the device 400 after extubation. The opening 414 at the superior end 414′ is shown as having a reduced diameter relative to the opening 415 at the superior end 415′, wherein the opening 414′ is sized to closely receive the breathing tube 106 therethrough, such as in a line-to-line or slightly loose fit, while the opposite opening 415 is enlarged to receive the increased size of the breathing tube 106 at its inferior end, such as a head of a LM or inflationary member on a ETT. One of skill in the art would readily appreciate that the sleeve could be secured to the ETT or LM and/or sealed at its inferior end 415′ using any of a number of fastening components. Merely by way of example, some of the fastening components that can be used include buttons, Velcro, adhesive tape, glue, clamps, string, malleable metal ties, magnets and the like.

FIG. 8A depicts, in accordance with an embodiment of the invention, a BFG 500 with a flexible sleeve wall 516 contained within a rigid or semi-rigid annulus 518, also referred to as hoop or ring, prior to use and prior to extension. In addition to having a plurality of tags 517 for applying finger pressure against the patient's face during extubation, along with an optional ridge 503 that can also be pushed against the patient's face during extubation by the caregiver's fingers. The device 500 has at least one, and shown as a plurality of superior end fasteners, also referred to as adhesive tags 519, extending from the superior end of the sleeve wall 516. The tags 519 can be used to fix the sleeve wall 516 to a breathing tube 106 after intubation (insertion of the tube 106 through the collapsed sleeve wall 516 and into the patient), and then, during extubation, the sleeve wall 516 will automatically be caused to unfold and extend around the tube 106 upon extubation, as shown in FIG. 8B.

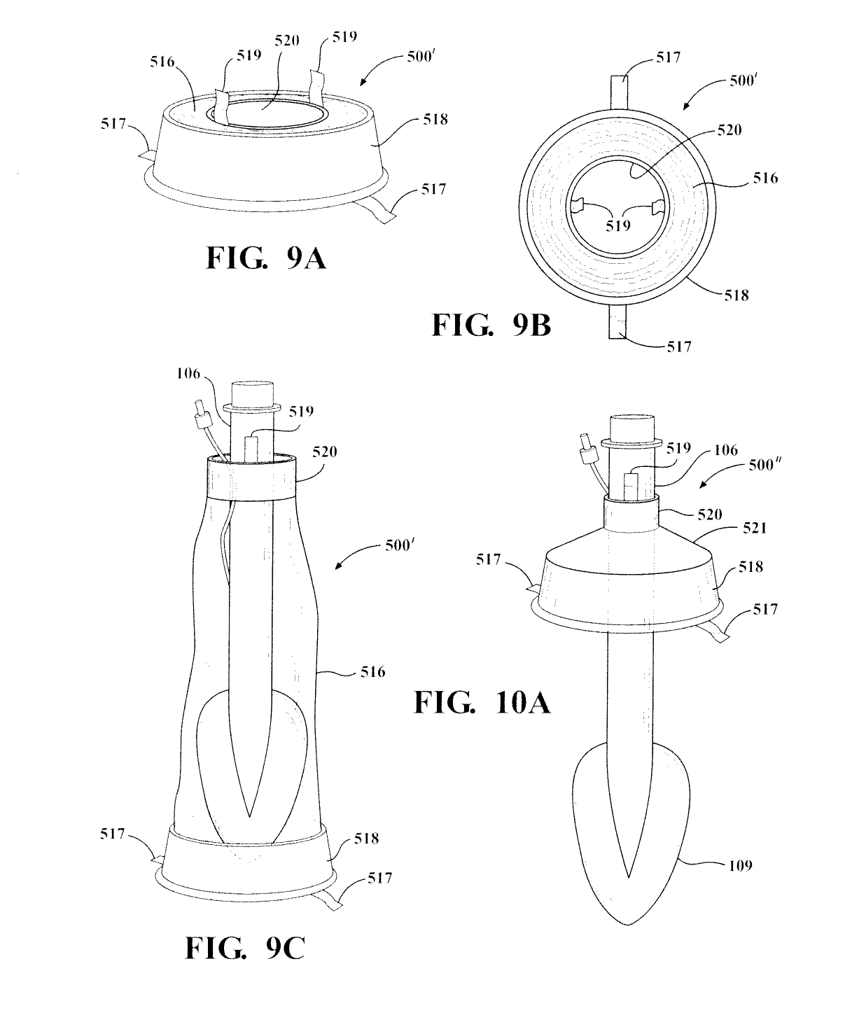

FIG. 9A depicts, in accordance with an embodiment of the invention, a BFG 500′ with a flexible sleeve wall 516 contained between a rigid or semi-rigid second annular ring, also referred to as outer annulus 518, also referred to as hoop or ring, and a rigid or semi-rigid first annular ring, also referred to as inner annulus 520, also referred to as hoop or ring prior to use and prior to extension. The concentric rings 518, 520 are concentric with one another, wherein the first ring 520 has a reduced diameter relative to the second ring 518, and the flexible wall 516 is attached to the outer ring 518 at one end and to the inner ring 520 at an opposite end. The outer ring 518 is configured similarly as discussed above for the device 500, including tags 417, and the inner ring 520 includes tags 519 to function as described above for the device 500. FIG. 9B depicts a top view of the inner ring 520 separated from the outer ring 518 and connected thereto by the extended sleeve wall 516. FIG. 9C depicts an end of a breathing tube 106 contained within the BFG 500′ upon extubation of the BFG 500′ from the patient.

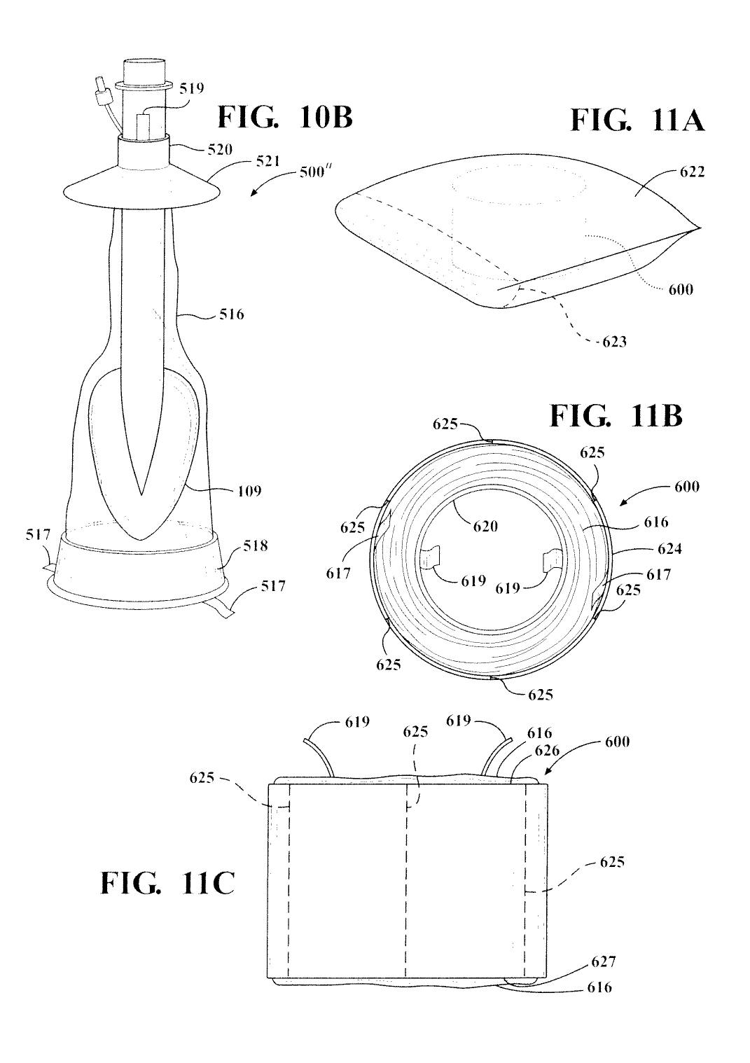

FIG. 10A depicts, in accordance with an embodiment of the invention, a BFG 500″ similar to the previously discussed BFG 500′, including a flexible sleeve wall 516 contained between a pair of rigid or semi-rigid outer annulus 518, 520. The BFG 500″ is shown as being assembled on a breathing tube 106, shown as a LM type breathing tube, by way of example and without limitation. The ring 520 includes a radially outwardly extending cone section 521 sized to extend to the periphery of the ring 518 when in the collapsed configuration of FIG. 10A. As such, the cone 521 covers the sleeve wall 516, thereby providing protection thereto prior to use. During extubation, as shown in FIG. 10B, the breathing tube 106 is fixed to the small ring 520 via fastener tags 519, and thus, the small ring 520 and cone 521 move conjointly with the breathing tube 106 as it is pulled through the larger ring 518 which is preferably attached to the patient via fastener tags 517. When extubation is complete, the head 109 of the LM is trapped and fully contained in the BFG 500″, wherein the sleeve wall 516 extends nearly the entire length of the breathing tube 106.

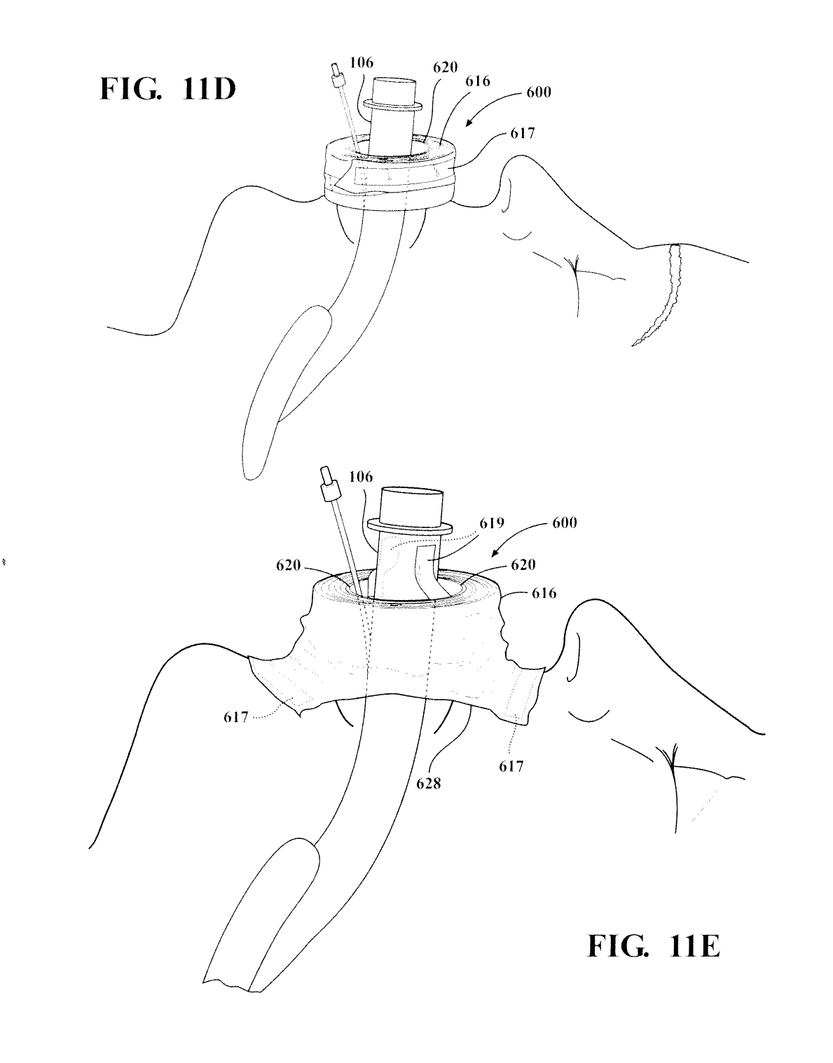

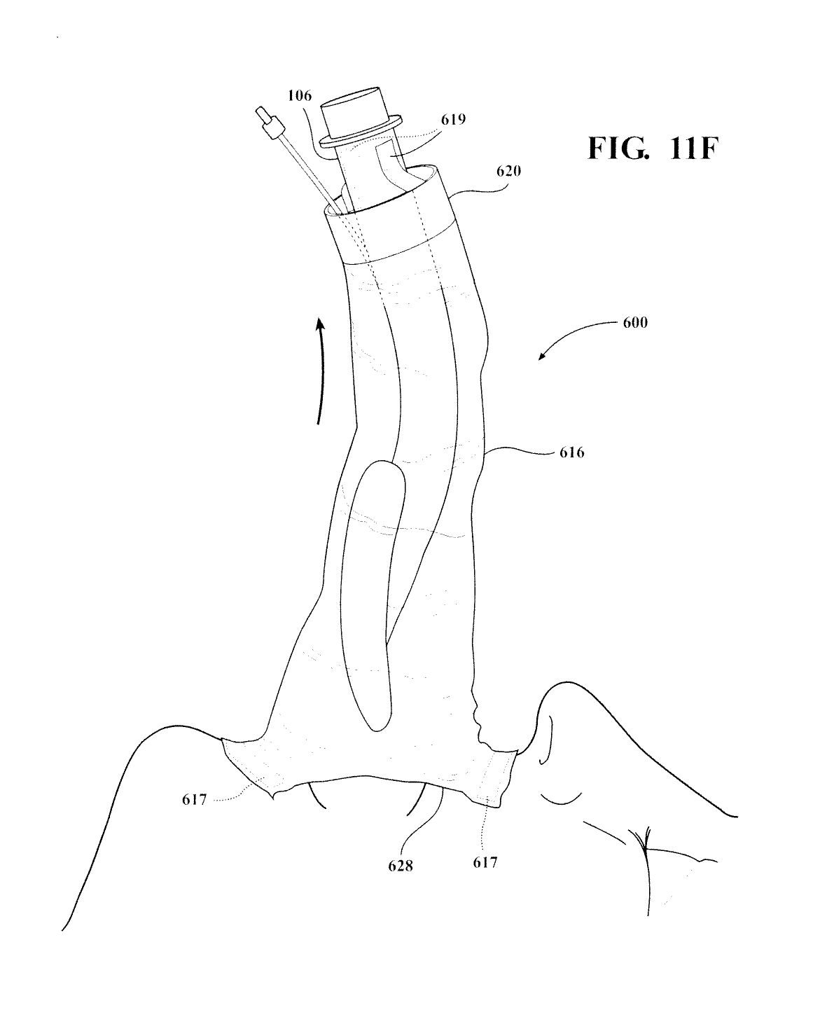

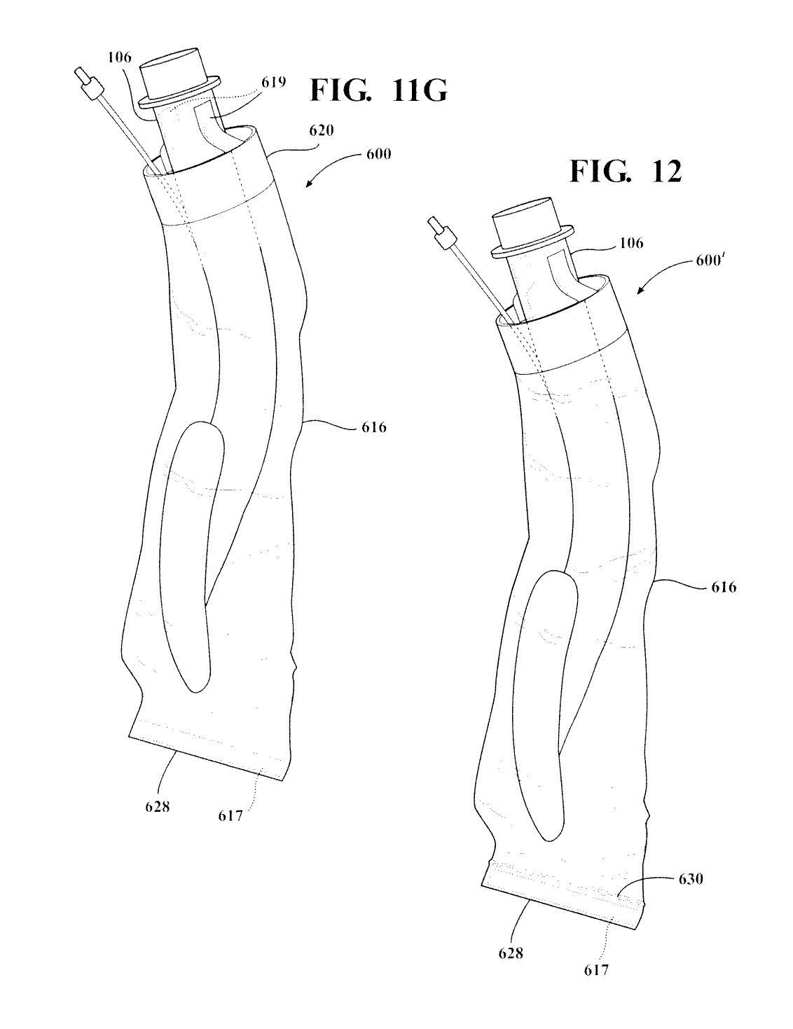

FIG. 11A depicts, in accordance with an embodiment of the invention, a BFG 600 packaged in a sterile pouch 622. The pouch 622 has a perforated side 623 to allow ready access to the BFG 600 as desired. As shown in FIG. 11B, upon removal of the BFG 600 from the pouch 622, the BFG 600 is remains contained in a containment band 624, such as an annular band of polymeric material, for example. To facilitate removal of the band 624, the band 624 can include perforations 625 extending between opposite ends 626, 627, such that a section of the band 624 can be readily torn to selectively open and remove the band 624 from the BFG 600, as desired, while a breathing tube 106 is extended through a core of the BFG 600. In use, the breathing tube 106 is disposed through the BFG 600 and inserted into the patient as prescribed by the breathing tube instructions. As shown in FIG. 11D, the BFG 600 is placed against the patient's mouth and then, as shown in FIG. 11E, an inferior end 628 of the flexible, collapsed wall 616 is fixed to the patients face, such as via adhesive tags 617. The adhesive tags 617 are shown as being diametrically opposite one another, for example, and initially have a release paper thereon, which is selectively removed to allow the tags 617 to be adhered to the patient. Further, at least one, and shown as a pair of adhesive tags 619 are adhered to a side wall of the breathing tube 106 upon inserting the breathing tube into the patient. The adhesive tags 619 are fixed to an upper annular rigid or semi-rigid ring 620, as discussed above for BFG 500′. As such, upon extubation of the BFG 600, the sleeve wall 616 is automatically unfolded about the breathing tube 106 to contain any fluid and debris inside the sleeve wall 616. Then, as shown in FIG. 11G, upon completely removing the breathing tube 106 from the patient, the end 628 is seal closed, such as by adhering the tags 617 to one another. Accordingly, the breathing tube is protected from contaminating the surgical theatre as well as all those persons present, and the entire BFG and breathing tube can be readily discarded.

FIG. 12 depicts, in accordance with an embodiment of the invention, a BFG 600′ similar to the BFG 600, however, in addition to the adhesive tags 617 at the inferior end 628, the wall 616 includes a closure mechanism 630 adjacent the inferior end 628, shown as being immediately above the adhesive tags 617. The closure mechanism 630 is shown as a tongue and groove type seal lock, often referred to as a zip-lock type seal. Accordingly, upon extubation of the breathing tube 106 within the BFG 600′, the closure mechanism can be sealed to reliably contain any fluid and debris within the sealed wall 616.

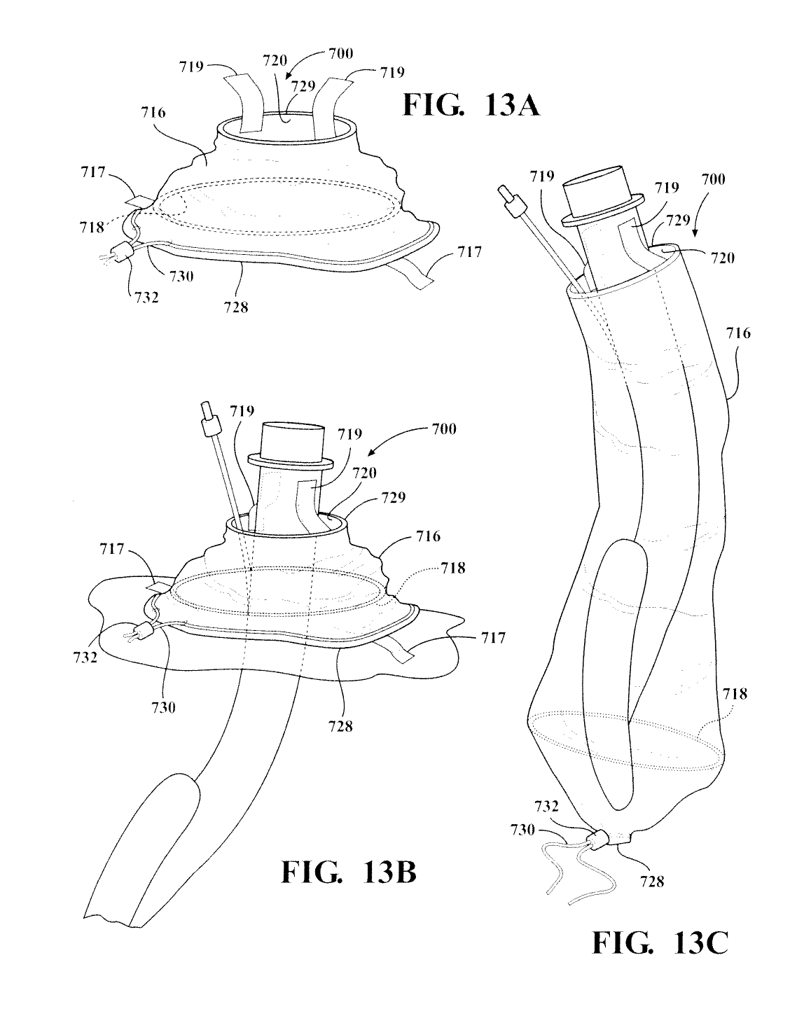

FIG. 12 depicts, in accordance with an embodiment of the invention, a BFG 700 having a flexible tubular sleeve wall 716 extending between opposite, open inferior and posterior ends 728, 729, wherein, as with all embodiments, the length of the wall 716 can be provided having any desired length, depending on the application requirements. As with embodiment of FIG. 9, the BFG 700 includes a pair of support rings 718, 720, wherein the upper support ring 718, however, rather than the lower support ring 718 being immediately adjacent the inferior end 728, the lower support ring 718 is spaced downwardly from the inferior end 728 a sufficient distance to provide a sufficient length of the wall 716 to be cinched to a closed configuration by a closure mechanism 730, as shown in FIG. 13C. The closure mechanism 730 is located immediately adjacent the inferior end 728, and is shown as a draw string type mechanism having a releasable, slidable locking member 732 to maintain the draw string in the cinched position, when desired. As shown in FIG. 13B, the BFG 700 includes lower adhesive tags 717 for fixing the lower end 728 to a patient's face and upper adhesive tags 719 for fixing the upper end 729 to a breathing tube 106.

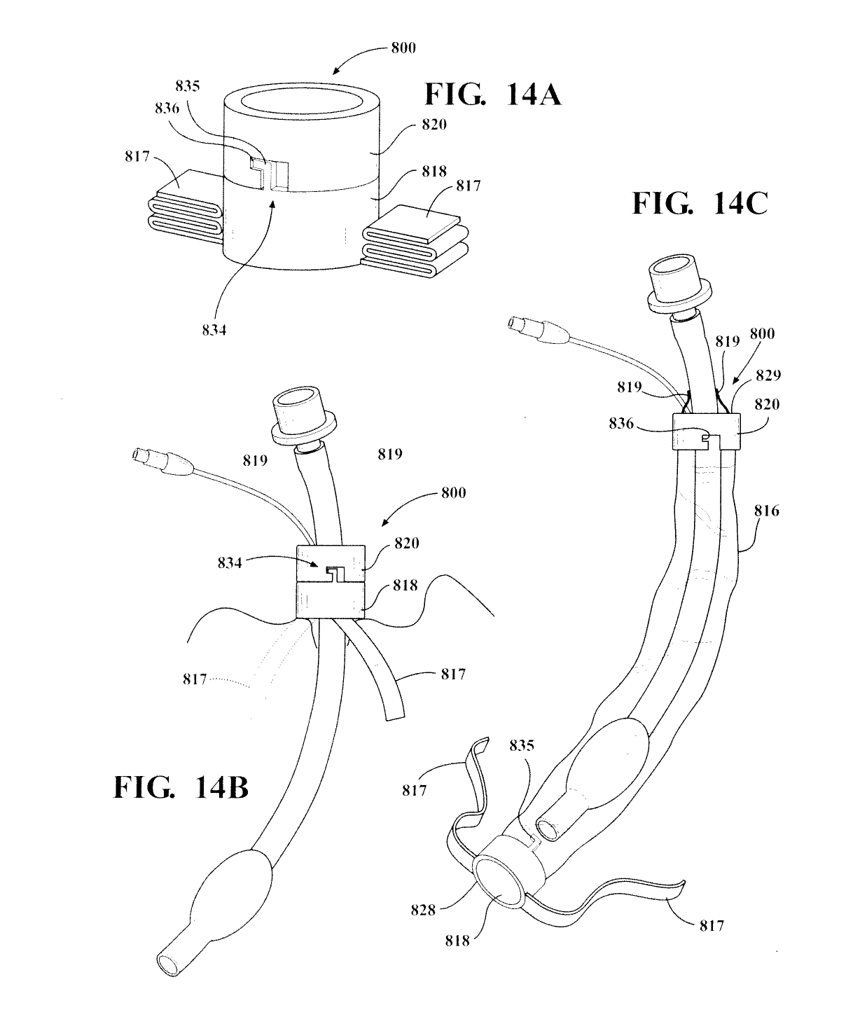

FIG. 14 depicts, in accordance with an embodiment of the invention, a BFG 800 having a flexible tubular sleeve wall 816 (FIG. 14C) extending between opposite, open inferior and posterior ends 828, 829. The BFG 800 includes a pair of support rings 818, 820 immediately adjacent respective inferior and posterior ends 828, 829. As shown in FIG. 14B, the BFG 800 includes lower inferior end adhesive tags 817 for releasably fixing the lower end 828 to a patient's face while the upper end includes upper superior end adhesive tags 819 for fixing the upper end 829 to a breathing tube 106. As shown in FIG. 14A, when in a collapsed configuration, the lower and upper rings 818, 820 can be maintained in abutment with one another via a closure mechanism 834, thereby preventing inadvertent separation of the rings 818, 820 from one another, which in turn, facilitates protecting the sleeve wall 816 from being inadvertently exposed. The closure mechanism is shown here as having a male member 835 and female member 836 configured for releasable locking engagement with one another. The male member 835 is shown as being generally L-shaped for an interlocking fit within the female member 836, which is similarly, negatively shaped as a L-shaped notch. Of course, one skilled in the art will contemplate, in view of this description, that other mechanisms are possible, such as a frangible tab extending between the rings 818, 820, or otherwise.

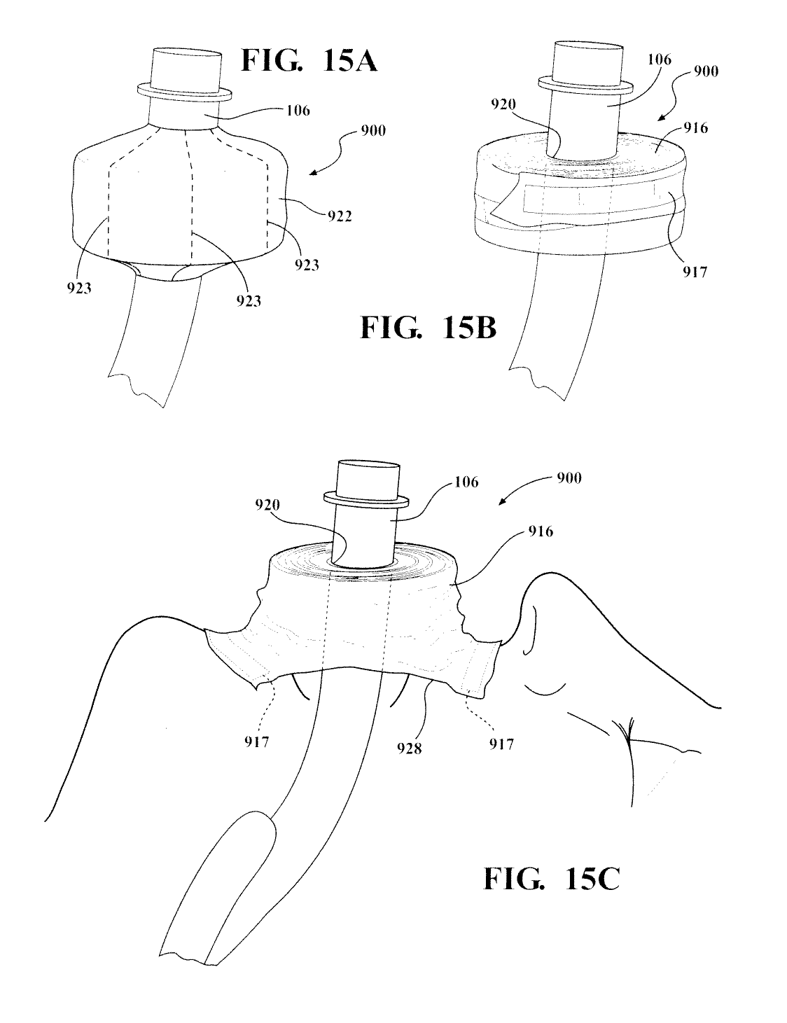

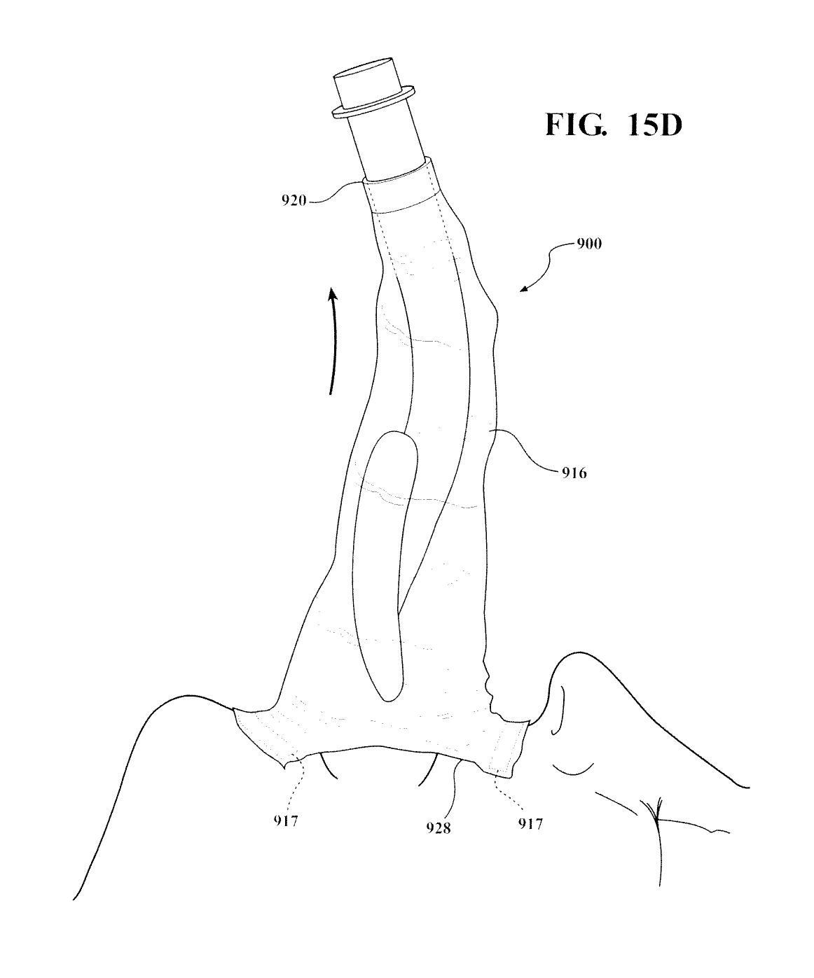

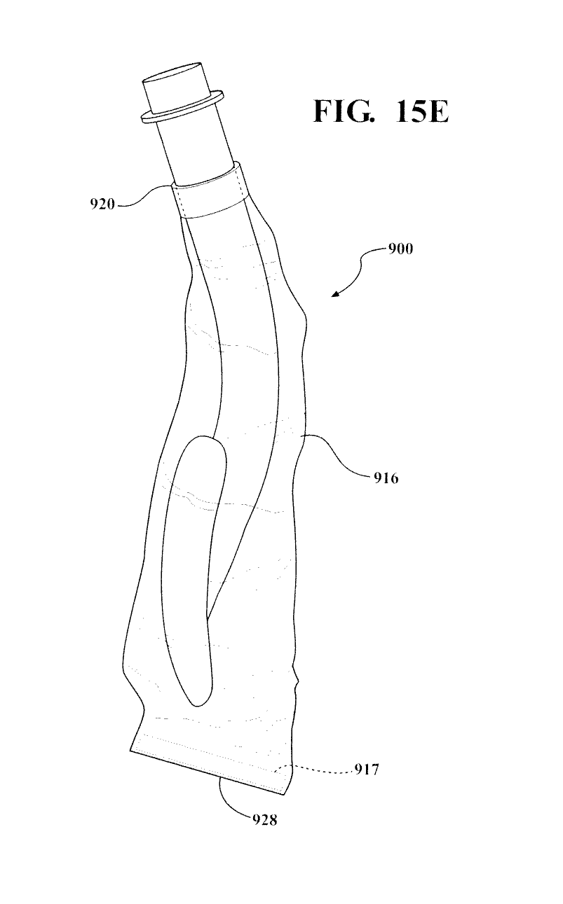

FIG. 15A depicts, in accordance with an embodiment of the invention, a BFG 900 packaged in a sterile wrapper 922, wherein the BFG is integrated as an assembly with a tubular surgical device, such as that of a breathing tube of a LM or ETT device, or a bronchoscope or catheter, by way of example and without limitation. The wrapper 922 is configured to be readily removed, when desired, and is shown as having a perforated side 923 to allow ready access to the BFG 900 contained in sterilized fashion therein, as desired, as shown in FIG. 15B. As shown in FIG. 15C, by way of example and without limitation, the tubular surgical device assembled with the BFG 900 is shown as a breathing tube 106 disposed through the BFG 900 and inserted into the patient as prescribed by the breathing tube instructions. The BFG 900 is placed against the patient's mouth and then an inferior end 928 of the flexible, collapsed wall 916 is releasably fixed to the patients face, such as via adhesive tags 917. The BFG 900 is fixed as assembly in manufacture of the surgical device via an upper annular rigid or semi-rigid ring 920. As such, during extubation of the BFG 900, as shown in FIG. 15D, the sleeve wall 916 is automatically caused to be telescopically unfolded about the breathing tube 106 to contain any fluid and debris inside the sleeve wall 916. Then, as shown in FIG. 15E, upon completely removing the breathing tube 106 from the patient, the end 928 is seal closed, such as by adhering the adhesive tags 917 to one another, by way of example and without limitation. Accordingly, the breathing tube 106 is protected from contaminating the surgical theatre as well as all those persons present, and the entire BFG and breathing tube assembly can be readily discarded.

In the various embodiments discussed herein, the flexible sleeve walls described herein can be made of plastic, such as a clear flexible polymeric material. One of skill in the art would readily appreciate that numerous other materials could also be used, if desired. Merely by way of non-limiting examples, the sleeves of the inventive devices described herein can be made of Tyvek, cloth, reinforced paper, foil, thin rubber, polyurethane and the like.

One of skill in the art would readily appreciate that both the removably and permanently attached BFG devices described herein can be adapted for use with an endoscope or a urinary catheter. These BFG devices can be used to place and/or remove either an endoscope or a urinary catheter in order to reduce the likelihood of a caregiver's exposure to a patient's bodily fluids.

Therefore, in some embodiments, the invention teaches a BFG that can be permanently or removably attached to an endoscope device of the type used to perform a colonoscopy. In some embodiments, the BFG adapted for use with an endoscope has a flexible sleeve type design, as described herein. In certain embodiments, the BFG is a “dual” or “single” ring type device, as described herein and shown in the Figures. In some embodiments, the BFG is configured with wings and/or tags and/or adhesive and/or closure components, as described herein. In certain embodiments the BFG includes a stabilization ring, as described herein. One of skill in the art would readily appreciate that in sleeve type BFGs, configured for use with an endoscope of the type used for performing a colonoscopy, the sleeve of the BFG is of a sufficient length that it can be extended from a region of the endoscope located outside of the patient's body on one end, to the patient's buttocks on the other end. In certain embodiments, the inferior end of the sleeve of the BFG is configured to interact with a patient's buttocks while surrounding the patient's anus, in the same manner the BFGs previously described herein interact with a patient's face and surround a patient's mouth. In some embodiments, the opening in the BFG device is of a sufficient size to allow an endoscope used for a colonoscopy to pass through.

In certain embodiments, the invention teaches a BFG that can be permanently or removably attached to a urinary catheter. Merely by way of non-limiting example, the urinary catheter can be a Foley catheter. In some embodiments, the BFG adapted for use with a catheter has a sleeve type design, as disclosed herein. In certain embodiments, the BFG is a dual ring or single ring device, as described herein. In some embodiments, the device is configured with wings and/or tags and/or adhesive and/or closure components, as described herein. In certain embodiments, the device includes a stabilization ring as described herein. One of skill in the art would readily appreciate that in BFG devices adapted for use with a urinary catheter, the sleeve of the BFG is of a sufficient length that it can be extended from a region of the catheter located outside of the patient's body on one end, to the pelvic region surrounding the patient's urethra on the other end. In certain embodiments, the sleeve of the BFG is configured to interact with a patient's pelvic region and surround a patient's urethra in the same manner in which it interacts with a patient's face and surrounds a patient's mouth in other embodiments described herein. In some embodiments, the opening in the BFG device is of a size that allows a urinary catheter to pass through.

In various embodiments, the invention teaches a kit for intubating and/or extubating a subject. In certain embodiments, the kit includes a BFG device described herein that can be fitted to the tube of an LM or ETT device, or pre-packaged as one unit, tube and BFG. In some embodiments, the kit includes an LM or ETT described herein with a BFG integrated thereon during the manufacturing process.

In various embodiments, the invention teaches a kit for placing an endoscope in and/or removing an endoscope from a subject. In certain embodiments, the kit includes a BFG described herein configured to be used with an endoscope device.

In various embodiments, the invention teaches a kit for placing a catheter in and/or removing a catheter from a subject. In various embodiments, the kit includes a BFG described herein, configured to be used with a catheter device.

The exact nature of the additional components configured in the inventive kit depends on its intended purpose. For example, in some embodiments, the kit is configured for veterinary applications, and the devices can be used in subjects such as, but not limited to, farm animals, domestic animals, and laboratory animals. In some embodiments, the kit is configured for the purpose of intubating and/or extubating human subjects. In some embodiments, the kit is configured for the purpose of placing an endoscope in and/or removing an endoscope from a subject. In some embodiments, the kit is configured for the purpose of placing a urinary catheter in and/or removing a urinary catheter from a subject.

Instructions for use may be included in the kit. “Instructions for use” typically include a tangible expression describing the technique to be employed in using the components of the kit to effect a desired outcome, such as to intubate or extubate a patient. Optionally, the kit also contains other useful components, such as, lubricants, adhesives, disposable biological material containers or other useful paraphernalia as will be readily recognized by those of skill in the art.

The materials or components assembled in the kit are typically contained in suitable packaging material(s). As employed herein, the phrase “packaging material” refers to one or more physical structures used to house the contents of the kit, such as inventive BFG devices. The packaging material is constructed by well-known methods, preferably to provide a sterile, contaminant-free environment. The packaging materials employed in the kit can be those customarily utilized in human medicine or veterinary medicine. As used herein, the term “package” refers to a suitable solid matrix or material such as plastic, paper, foil, and the like, capable of holding the individual kit components. Thus, for example, a package can be one or more plastic containers used to contain one or more of the inventive devices described herein. The packaging material generally has an external label which indicates the contents, dimensions and/or purpose of the kit and/or its components. For example, the label may indicate that a specific device is to be used on a patient of a specific size, within a specific age range, or of a specific sex.

In various embodiments, the invention teaches a method of removing an LM or ETT device from a patient, including (1) using any one of the BFGs described herein that are configured to be used in conjunction with an existing LM or ETT device, and (2) removing the LM or ETT device from the patient. In some embodiments, the invention teaches a method of placing an LM or ETT device in a patient, including (1) using any of the BFGs described herein that are configured to be used in conjunction with an existing LM or ETT device, and (2) placing the LM or ETT device in the patient. In other embodiments, the invention teaches a method of removing an LM or ETT device from a patient, wherein the LM or ETT device includes a BFG described herein integrated thereon. In yet other embodiments, the invention teaches a method of placing an LM or ETT device in a patient, wherein the LM or ETT device includes a BFG described herein integrated thereon.

In various embodiments, the invention teaches a method of removing an endoscope device from a patient, including (1) using any one of the BFGs described herein that are configured to be used in conjunction with an endoscope device, and (2) removing the endoscope device from the patient. In some embodiments, the invention teaches a method of placing an endoscope device in a patient, including (1) using any of the BFGs described herein that are configured to be used in conjunction with an endoscope device, and (2) placing the endoscope device in the patient.

In various embodiments, the invention teaches a method of removing a urinary catheter device from a patient, including (1) using any one of the BFGs described herein that are configured to be used in conjunction with a urinary catheter device, and (2) removing the urinary catheter device from the patient. In some embodiments, the invention teaches a method of placing a urinary catheter device in a patient, including (1) using any of the BFGs described herein that are configured to be used in conjunction with a urinary catheter device, and (2) placing the urinary catheter device in the patient.

One skilled in the art will recognize many methods and materials similar or equivalent to those described herein which could be used in the practice of the present invention. Indeed, the present invention is in no way limited to the methods and materials described.

The following examples are for illustrative purposes only and are not intended to limit the scope of the disclosure or its various embodiments in any way.

### EXAMPLES

### Example 1

### Extubation Using a BFG Configured to Fit a LM Device

Extubation of an LM from a patient can be accomplished using the device shown in FIG. 2. A caregiver places the opening in the inferior end of the BFG over the breathing tube portion of the LM and slides the BFG all the way down the breathing tube until the BFG rests against the skin of the patient's mouth. The caregiver then extracts the portion of the LM 106 positioned inside of the patient through the hole in the center of the BFG 100, while the sponge material inside the BFG 100 is compressed against the tube 106 and the inferior surface of the BFG is simultaneously pressed gently against the patients face. The caregiver continues extracting the LM until the entire device, including the head (cuff) 109 is completely removed from the patient's mouth and through the BFG.

### Example 2

### Extubation of an LM Using a Single Ring Type BFG

Extubation of an LM from a patient can be accomplished using the BFG device 600 shown in FIGS. 8A and 8B. A caregiver places the opening in the inferior end of the BFG over the breathing tube portion of the LM 106. Next, the caregiver secures the tape 519 on the superior portion of the sleeve 516 to the tube 106. The caregiver then withdraws the tube 106 from the patient's mouth, while pressing the inferior end of the support ridge 503 against the patient's face, until the entire length of the LM, formerly inside the patient, is removed into the sleeve 500.

### Example 3

### Extubation Using an ETT with an Integrated BFG

Extubation of an ETT device from a patient can be accomplished using the ETT device 800 with an integrated and pre-packaged BFG shown in FIG. 14B. First, the caregiver removes an outer wrapper to gain access to the BFG 800 portion of the device. Next, the caregiver pulls down on the inferior ring 818 until the stabilization wings 817 contact the patient's face. The ETT device is then extracted from the patient's mouth into the sleeve 816.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A bodily fluids guard for containing fluid and debris on a tubular surgical device extubated from a patient, comprising:
a flexible tubular wall extending between an open superior end and an open inferior end; and
at least one inferior end fastener extending from said inferior end, said at least one fastener having a surface adapted for releasable fixation to a patient.

2. The bodily fluids guard of claim 1 further including at least one superior end fastener extending from said superior end, said at least one superior end fastener having a surface adapted for fixation to the tubular surgical device.

3. The bodily fluids guard of claim 2 wherein said at least one inferior end fastener and said at least one superior end fastener have self-adhesive surfaces.

4. The bodily fluids guard of claim 2 wherein said at least one inferior end fastener is configured to seal said open inferior end.

5. The bodily fluids guard of claim 4 wherein said inferior end fastener is a self-adhesive member.

6. The bodily fluids guard of claim 4 wherein said inferior end fastener is a zip lock member.

7. The bodily fluids guard of claim 4 wherein said inferior end fastener is a draw string.

8. The bodily fluids guard of claim 1 further comprising a first annular ring of semi-rigid material adjacent said superior end, said at least one superior end fastener extending from said first annular ring.

9. The bodily fluids guard of claim 8 further comprising a second annular ring of semi-rigid material adjacent said inferior end, said at least one inferior end fastener extending from said second annular ring.

10. The bodily fluids guard of claim 9 wherein said first annular ring has a reduced diameter relative to said second annular ring.

11. The bodily fluids guard of claim 10 wherein said first annular ring has a cone region extending radially outwardly therefrom toward said second annular ring.

12. The bodily fluids guard of claim 1 wherein said first annular ring and second annular ring are releasably coupled to one another.

13. The bodily fluids guard of claim 12 wherein said first and second annular rings have mating male and female members for releasable attachment to one another.

14. The bodily fluids guard of claim 1 wherein the tubular surgical device is one of a LM, ETT, bronchoscope and catheter.

15. The bodily fluids guard of claim 14 wherein said bodily fluids guard is fixed to the tubular surgical device as an integrated assembly.

16. A bodily fluids guard for containing fluid and debris on a tubular surgical device extubated from a patient, comprising:
a superior end;
an inferior end;
an outer layer;
an inner layer having a hollow core; and
wherein said inner and outer layers are connected to one another and said inner layer includes a compressible and absorbent material, said outer layer includes a flexible material, and said hollow core is configured to receive the tubular surgical device.

17. The bodily fluids guard of claim 16 further including bristles extending radially inwardly into said hollow core.

18. The bodily fluids guard of claim 16 further including at least one grasping feature extending radially outward from said outer layer.

19. The bodily fluids guard of claim 16 wherein said outer layer is plastic.

20. The bodily fluids guard of claim 16 wherein the tubular surgical device is one of a LM, ETT, bronchoscope and catheter.

21. A method of containing fluid and debris on a tubular surgical device extubated from a patient, comprising:
providing flexible tubular wall extending between an open superior end and an open inferior end in a lengthwise collapsed state;
intubating the tubular surgical device in the patient with the flexible tubular wall disposed about the tubular surgical device;
releasably fastening the inferior end of the flexible tubular wall to the patient; and
extubating the tubular surgical device from the patient and simultaneously lengthening the tubular wall to surround the tubular surgical device.

22. The method of claim 21 further including fastening the superior end to the tubular surgical device prior to extubating.

23. The method of claim 22 further including fastening the inferior end to the patient with a self-adhesive and fastening the superior end to the tubular surgical device with a self-adhesive.

24. The method of claim 22 further including providing the flexible tubular wall and the tubular surgical device as an assembly fixed to one another.

25. The method of claim 21 further including sealing the open inferior end closed after extubating the tubular surgical device with a fastener adjacent the open inferior end.

26. The method of claim 21 wherein the tubular surgical device is one of a LM, ETT, bronchoscope and catheter.

26. The method of claim 25 wherein said bodily fluids guard is fixed to the tubular surgical device as an integrated assembly.
